(19) United States
(12) Patent Application Publication
(10) Pub. No.: US 2025/0297309 A1
(43) Pub. Date: Sep. 25, 2025

(54) METHODS FOR AMYOTROPHIC LATERAL SCLEROSIS

(71) Applicant: Thomas Jefferson University, Philadelphia, PA (US)

(72) Inventors: Isidore Rigoutsos, Astoria, NY (US); Phillipe Loher, Davidson, NC (US)

(21) Appl. No.: 18/715,608

(22) PCT Filed: Dec. 8, 2022
(86) PCT No.: PCT/US2022/081183

§ 371 Date: May 31, 2024

Related U.S. Application Data
(60) Provisional application No. 63/287,312, filed on Dec. 8, 2021.

(51) Int. Cl. C12Q 1/6883; A61K 31/428; A61K 45/06

(52) U.S. Cl. CPC C12Q 1/6883; A61K 31/428; A61K 45/06; C12Q 2600/112; C12Q 2600/118; C12Q 2600/158

(57) ABSTRACT

Disclosed herein are methods for treating or preventing amyotrophic lateral sclerosis (ALS) in a subject in need thereof. The method comprises isolating RNAs from a sample obtained from the subject; characterizing the RNAs and their relative abundance in the sample to identify a signature, wherein the signature is indicative of presence or absence of ALS; and administering to the subject a treatment for ALS. Also disclosed herein are methods for diagnosis of ALS, determining the stage of ALS and predicting speed of progression of ALS, using short RNA signatures associated with diagnosis, stage or progression speed of ALS.

### CROSS-REFERENCE TO RELATED APPLICATIONS

The present application claims priority under 35 U.S.C. § 119(e) to U.S. Provisional Patent Application No. 63/287,312, filed December 8. 2021, which is incorporated herein by reference in its entirety.

### SEQUENCE LISTING

The ASCII text file named “205-7080WOJ(00314) Seq Listing. xml” created on Dec. 8, 2022, comprising 58,205 Kbytes, is hereby incorporated by reference in its entirety.

### BACKGROUND

Amyotrophic lateral sclerosis (ALS), also referred to as Lou Gehrig's disease is neurodegenerative disease that results in the progressive loss of motor neurons that control voluntary muscles.

Currently, there is no single test that can provide definitive diagnosis of amyotrophic lateral sclerosis (ALS). Rather, ALS is diagnosed based on a detailed history of the symptoms observed by a physician as well as a series of tests to, among others, rule out other diseases. Accordingly, there is a need for novel methods to diagnose ALS so that the disease can be treated al early stages.

Furthermore, ALS progresses differently in individuals. It is difficult to determine the stage or the speed of progression of the disease in an individual. Accordingly, there is a need for novel method to evaluate in individuals the stage and speed of progression of ALS.

The present invention addresses the above needs.

### SUMMARY

In some aspects, the present invention is directed to a method for treating or preventing amyotrophic lateral sclerosis (ALS) in a subject in need thereof.

In some embodiments, the method comprises: isolating fragments of short RNAs from a sample obtained from the subject: characterizing the short RNAs and their relative abundance in the sample to identify a signature, wherein when the signature is indicative of a presence or absence of ALS; and if the identified signature indicates a presence of ALS, administering to the subject a treatment of ALS. In some aspects, the present invention is directed to a method of estimating a speed of progression of amyotrophic lateral sclerosis (ALS) in a subject. In some embodiments, the method comprises: isolating fragments of short RNAs from a sample obtained from the subject, characterizing the short RNAs and their relative abundance in the sample to identify a signature, wherein when the signature is indicative of a progression speed of ALS; and determining the speed of ALS progression in the subject based on the identified signature.

In some aspects, the present invention is directed to a method of determining a stage of amyotrophic lateral sclerosis (ALS) in a subject. In some embodiments, the method comprises: isolating fragments of short RNAs from a sample obtained from the subject; characterizing the short RNAs and their relative abundance in the sample to identify a signature, wherein when the signature is indicative of a stage of ALS; and determining the stage of the ALS in the subject based on the identified signature.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawing s. It should be understood, however, that the instant specification is not limited to the precise arrangements and instrumentalities of the embodiments shown in the drawings.

FIGS. 1A-1J: non-limiting short RNA sequences that are associated with ALS patient survival in a statistically significant manner, in accordance with some embodiments. The sequences shown in FIGS. 1A-1J are also listed below:

GUCGACGUAUAGGGUGUG (SEQ ID NO: 2328)
GCGGGCGUCGUUCAAUGGUAGGACCUGAGCU (SEQ ID NO: 2758)
UGCAAGUCGAACGGUA (SEQ ID NO: 2522)
GUAAUGGCGGGAACUCUGGACAGACUGCCU (SEQ ID NO: 1194)
GAUUUCCCCUUGGGGUUGUAGGACCA (SEQ ID NO: 1120)
UAAAGUGCUUAUAGUGCAGGUAG (SEQ ID NO: 1369)
UCUUUGGUUAUCUAGCUGUAU (SEQ ID NO: 3004)
GCCGUGAUCGUAUAGUGGUUAGUACUCUGC (SEQ ID NO: 1236)
CGACUCUUAGCGGUGGAUCACUCGGCUCGUGCGUCGAUGAAGA
(SEQ ID NO: 727)
CGCGACCUCAGAUCAGACGUGGCGACCCGCU (SEQ ID NO: 758)

### DETAILED DESCRIPTION

The following disclosure provides many different embodiments, or examples, for implementing different features of the provided subject matter Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is included in and/or selected from a list of recited elements or components, it should be understood that the element or component can be any one of the recited elements or components and can be selected from a group consisting of two or more of the recited elements of components.

In the methods described herein, the acts can be carried out in any order, except when a temporal of operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms or “the” are used to include one or more than one unless the context clearly dictates otherwise. The term “or” is used to refer to a nonexclusive “or” unless otherwise indicated. The statement “at least one of A and B” or “at least one of Å of B” has the same meaning as “A. B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods

### Methods for Diagnosing, Treating, or Preventing Amyotrophic Lateral Sclerosis (ALS)

The study described herein (“the present study”) analyzed short RNA datasets from two public repositories. The first repository comprises datasets generated from plasma samples of ALS patients and healthy controls, and the second repository comprises datasets generated from serum samples of ALS patients and healthy controls. The analyses show that a set of short RNAs as well as their level In the samples correlate with ALS, and therefor can be used for diagnosing ALS, as well as for treating or preventing the disease by providing guidance for the administration of medications and therapies.

Accordingly, in some aspects, the present invention is directed to a method for diagnosing ALS.

In some embodiments, the method for diagnosing ALS comprises isolating short RNAs from a sample obtained from the subject; and characterizing the short RNAs and their relative abundance in the sample to identify a signature, wherein when the signature is indicative of a presence or absence of ALS.

In some aspects, the present invention is directed to a method for treating, ameliorating and/or preventing ALS.

In some embodiments, the method for treating or preventing ALS comprises: isolating short RNAs from a sample obtained from the subject; characterizing the short RNAs and their relative abundance in the sample to identify a signature, wherein when the signature is indicative of a presence or absence of ALS; and if the identified signature indicates a presence of ALS, administering to the a treatment of ALS.

In some embodiments, the short RNA is an RNA molecule having a length of about 100 nucleotides or less, such as about 90 nucleotides or less, about 80 nucleotides or less, about 70 nucleotides or less, about 60 nucleotides or less, or about 50 nucleotides or less.

In some embodiments, the signature indicating the presence of ALS comprises at least one short RNA selected from the group consisting of RNAs set forth in SEQ ID NOs: 1-1679 and 65683-66986.

In some embodiments, the signature indicating the presence of ALS comprises at least one selected from the group consisting of: GCUGUGAUGGCCGAGUGG (SEQ ID NO:1222), GGGGAUGUAGCUCAGUGG (SEQ ID NO:1231), CGGGCCUGGUUAGUACUUGGAUGU (SEQ ID NO:32), and UCGGCUGUUAACCGAAAGGUUGGUGGU (SEQ ID NO:484).

In some embodiments, the signature comprises at least one short RNA selected from the group consisting of RNAs set forth in SEQ ID NOs: 1-711, 1481-1545, 65683-65745, 66521-66593, 66632-66674, and 66876-66986, and an increased level of the at least one short RNA In comparison to a baseline level (e.g., the level found in control subjects that do not have ALS) is indicative of a presence of ALS.

In some embodiments, the signature comprises at least one short RNA selected from the group consisting of RNAs set forth in SEQ ID NOs: 712-1480, 1546-1679, 65746-66520, 66594-66631, 66675-66875, and 66987-67130, and a decreased level of the at least one short RNA in comparison to a base line is indicative of a presence of ALS.

In some embodiments, the level of the at least one short RNA of the signature changes (such as increases or decreases) by about 2 folds or more, such as about 5 folds or more, about 10 folds or more, about 20 folds or more, about 50 folds or more, about 100 folds or more, about 200 folds or more, about 500 folds or more, or about 1000 folds or more, based on the base line level

In some embodiments, the sample is isolated from a cell, tissue or body fluid obtained from the subject.

In some embodiments, the sample is isolated from a body fluid, and the body fluid is a plasma, a serum, a cerebrospinal fluid, or combinations thereof.

In some embodiments, the treatment of ALS comprises a glutamate blocker, a muscle relaxant, a physical therapy, or combinations thereof.

Non-limiting examples of glutamate blocker useful for treating ALS include riluzole, and the like.

Non-limiting examples of muscle relaxant useful for treating ALS include baclofen, tizanidine, and the like.

The present study has discovered that short RNAs derived from some bacteria, such as, for example, those from the phylum Verumicrobia and the phylum Proteabacteria, are associated with ALS. Accordingly, in some embodiments, the method of treating or preventing ALS further comprises reducing and/or eliminating these bacteria in the body of the subject: Medications, such as antibiotics, for reducing and/or eliminating bacteria, such as Verumicrobia and/or Proteobacteria, are well known in the art and will not be described herein.

Accordingly, in some embodiments, the at least one of the signature indicatives of the presence of ALS identified in the sample comprises at least one short RNA originated from a bacterium, and wherein subject is further administered with a medication for an infection of the bacterium.

In some embodiments, the bacterium comprises at least one bacterium of the phylum Verumicrobia and/or at least one bacterium from the phylum Proteobacteria.

In some embodiments, the medication for the infection of the bacterium comprises an antibiotic.

In some embodiments, the subject is a mammal, such as a human.

### Methods for Estimating Speed of ALS Progression

The present study identified short RNAs in ALS patient samples that correlate with neurofilament light chain (NfL), which is a quantitative biomarker of ALS aggressiveness, as well as short RNAs that correlate with the rate of disease progression rate/velocity of ALSFRS (also referred to as the “slope”) These short RNAs, as well as their levels, can be used to estimate ALS aggressiveness.

Accordingly, in some aspects, the present invention is directed to a method for estimating a speed of progression of amyotrophic lateral sclerosis (ALS) in a subject.

In some embodiments, the method comprises: isolating short RNAs from a sample obtained from the subject; characterizing the short RNAs and their relative abundance in the sample to identify a signature, wherein when the signature is indicative of a progression speed of ALS; and determining the speed of ALS progression in the subject based on the identified signature.

In some embodiments, the short RNA is an RNA molecule having a length of about 100 nucleotides or less, such as about 90 nucleotides or less, about 80 nucleotides or less, about 70 nucleotides or less, about 60 nucleotides or less, or about 50 nucleotides or less.

In some embodiments, the signature comprises at least one short RNAs selected from the group set forth in SEQ ID NOs: 1680-2725. In some embodiments, an altered level of at least one short RNAs selected from the group set forth in SEQ ID NOs: 1680-2725 in comparison to a reference level (e.g., a level of the short RNAs in control subjects that do not have ALS) is Indicative of fast ALS progression.

In some embodiments, the sample is isolated from a cell, tissue or body fluid obtained from the subject.

In some embodiments, the sample is isolated from a body fluid, and the body fluid is a plasma, a serum, a cerebrospinal fluid, or combinations thereof.

In some embodiments, the subject is a mammal, such as a human.

### Methods for Determining Stage of ALS

The present study identified short RNAs in ALS patient samples that correlate with the stage of ALS, such as correlate with the predict time-to-death of the disease.

Accordingly, in some aspects, the present invention is directed to a method for estimating a stage of amyotrophic lateral sclerosis (ALS) in a subject.

In some embodiments, the method comprises isolating short RNAs from a sample obtained from the subject; characterizing the short RNAs and their relative abundance in the sample to identify a signature, wherein when the signature is indicative of a stage of ALS; and determining the stage of ALS progression in the subject based on the identified signature.

In some embodiments, the short RNA is an RNA molecule having a length of about 100 nucleotides or less, such as about 90 nucleotides or less, about 80 nucleotides or less, about 70 nucleotides or less, about 60 nucleotides or less, or about 50 nucleotides or less.

In some embodiments, the signature comprises at least one short RNA selected from the group consisting of RNAs set forth in SEQ ID NOs: 2726-650 In some embodiments, an altered level of at least one short RNA selected from the group set forth in SEQ ID NOs: 2726-65054 in comparison to a reference level (e.g., a level of the short RNAs in control subjects that do not have ALS) is indicative of the stage of ALS.

In some embodiments, the signature comprises at least one short RNAs selected from the group consisting of

(SEQ ID NO: 727)
CGACUCUUAGCGGUGGAUCACUCGGCUCGUGCGUCGAUGAAGA,
(SEQ ID NO: 758)
CGCGACCUCAGAUCAGACGUGGCGACCCGCU,
(SEQ ID NO: 1120)
GAUUUCCCCUUGGGGUUGUAGGACCA,
(SEQ ID NO: 1194)
GUAAUGGCGGGAACUCUGGACAGACUGCCU,
(SEQ ID NO: 1236)
GCCGUGAUCGUAUAGUGGUUAGUACUCUGC,
(SEQ ID NO: 1369)
UAAAGUGCUUAUAGUGCAGGUAG,
(SEQ ID NO: 2328)
GUCGACGUAUAGGGUGUG,
(SEQ ID NO: 2522)
UGCAAGUCGAACGGUA,
(SEQ ID NO: 2758)
GCGGGCGUCGUUCAAUGGUAGGACCUGAGCU,
and
(SEQ ID NO: 3004)
UCUUUGGUUAUCUAGCUGUAU.

In some embodiments, the sample is isolated from a cell, tissue or body fluid obtained from the subject.

In some embodiments, the sample is isolated from a body fluid, and the body fluid is a plasma, a serum, a cerebrospinal fluid, or combinations thereof.

In some embodiments, the subject is a mammal, such as a human.

### Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein

In this section, analyses of two collections of public data (RNA-seq) that were generated from patients with amyotrophic lateral sclerosis (ALS) and controls will be described. The analyses led to the identification of multiple short RNA molecules that individually or in combinations can be used to: 1) diagnose ALS; 2) determine how quickly the disease is progressing; and/or 3) serve as candidate therapeutic targets.

The RNA molecules belong to the following classes: a) miRNA isoforms, also referred to as isomiRs; b) tRNA-derived fragments, also referred to as tRFs; c) rRNA-derived fragments, also referred to as rRFs, and d) “Not-TRF-ISO-KRF,” short RNA that cannot be categorized as human isomiRs, tRFs or rRFs using exact matching.

The present study analyzed datasets from two public repositories. The first repository comprised datasets generated from plasma samples of ALS patients and healthy controls described in Magen et al., Circulating miR-181 is a prognostic biomarker for amyotrophic lateral sclerosis. Nature Neuroscience. 2021 (https://doi.org/10.1101/833079). The second repository comprised datasets generated from serum samples of ALS patients and healthy controls described in Dobrowolny et al, A longitudinal study defined circulating microRNAs as reliable biomarkers for disease prognosis and progression in ALS human patients, Cell Death Discov. 2021 (https://doi.org/10.1038/s41420-020-00397-6)

The Example section herein describes t present study in reference to several tables (Tables 1-9), which are shown after the “Enumerated Embodiments” section. any thymine (T) nucleotide recited in the nucleotide sequences disclosed in Tables 1-9 is intended to represent a uracil (U) nucleotide and is consistence with the WIPO Standard ST.26.

### Example 1: First of the Two Collections-GSE168714

### About the Data

The RNA-seq data of the first collection appear on NIH's Gene Expression Omnibus (GEO) under accession number GSE168714. Table 1 lists the datasets that the present study analyzed from this collection, their GEO and Sequence Read Archive (SRA) identifiers, and associated information

### Preprocessing of the Deep Sequencing Datasets

To help control for library amplification bias, random unique molecular identifiers (UMIs) were used during the preparation of the libraries from the samples comprising the GSE168714 collection. The UMIs are 12 nucleotides (nts) in length and are added to the sequencing libraries before any PCR amplification. UMIs are meant to tag the unique molecules during the adapter ligation step. Their incorporation in this instance is meant to help identify instances of biased amplification during the PCR step: by accounting for any bias that may be present, one can avoid potentially erroneous quantitation estimates for the sequenced molecules.

After sequencing and prior to quantifying the present molecules, this process needs to be reversed and the UMIs removed from the sequenced reads. To this end, the present study used a filtering strategy similar to what is described in Potla et al. “A bioinformatics approach to microRNA-sequencing analysis,” Osteoarthritis and Cartilage Open 2021 (https://doi.org/10.1016/j.ocarto.2020.100131). For the purpose of UMI-deduplication, the analysis leveraged the opensource “UMI-Tools” package, originally described in Smith et al. “UMI-tools: Modelling sequencing errors in Unique Molecular Identifiers to improve quantification accuracy” Genome Research 2017 (http://www.genome.org/cgi/doi/10.1101/gr.209601.116) Following a quality-assessment filtering step, the present study used the ‘extract’ command from the UMI-tools package to remove the 3 adapter, while allowing up to 2 mismatches, and to identify the UMI tag for each read. At this stage, any reads that did not contain a 3′ adapter were discarded. After identified the different UMI tags for each read, the package's ‘count top’ command was used to form clusters with shared tags and to remove duplicates (“de-duplication” step). The present study used the default arguments with count_tab (method=“directional”) that relies on Hamming distance to Identify identical and near-Identical UMI tags. Once the UMI-containing reads were deduplicated, only reads with length≥15 nts were kept for further processing.

### Mining of the Deep Sequencing Datasets

The present study preprocessed each raw dataset from Table 2 as outlined above. The present study profiled the short RNA in each dataset by focusing on four classes. The first three classes are: isomiRs; tRPs; and rRFs. The fourth class, Not-TRE-ISO-RRF, comprises all RNAs that cannot be labeled as human isomiRs, tRFs, or rRFs using exact sequence matching. Note that the Not-TRE-ISO-RRF class can comprise: unannotated isomiRs, tRFs, or rRFs; SNP- or mutation-containing isomiRs, tRFs, or rREs, repeat-element-derived fragments, i.e. short RNAs that map to repeat elements, other short RNA of human origin that cannot be mapped to the standard human genome assembly using exact sequence matching; isomiR, tRF, rRF or other types of short RNA do not originate in the human genome.

The present study used the following approaches to identify and quantify the short RNAs of each class:
isoMiRmap (Loher et al, “IsoMiRmap-fast, deterministic, and exhaustive mining of isomiRs from short RNA-seq datasets.” Bioinformatics 2021-https://doi.org/10.1093/bioinformatics/btab016) for isomiRs. Here, the present study sought isomiRs from the miRNAs contained in miRCarta Rel. 1. 1 described in Backes,C. et al. “miRCarta: a central repository for collecting miRNA candidates” Nucleic Acids Res. 2017 (https://doi.org/10.1093/mar/gkx851)
MINTmap (Loher et al. “MINTmap: fast and exhaustive profiling of nuclear and mitochondrial tRNA fragments from short RNA-seq data,” Sci Rep 2017-https://10.1038/srep41184) for tRFs. Here, the present study sought tRFs from the nuclear and mitochondrial tRNAs described in the MINTmap publication.
MINRmap (unpublished) for rRFs. Here, the present study sought tRFs that arise from the six rRNAs described in Cherlin et al. “Ribosomal RNA fragmentation into short RNAs (rRFs) is modulated in a sex-and population of origin-specific manner” BMC Biology 2020 . . . https://doi.org/10.1186/s12915-020-0763-0.
The present study labeled Not-TRP-ISO-RRF those reads that remained from the starting collection of sequenced reads after accounting for those reads that can be assigned to human isomiRs, tRFs, and rRFs using exact sequence matching. Since these reads are already UMI-deduplicated, the present study identified the unique sequences among them, calculated the copy number for each such sequence (=“abundance”), and assigned it to the corresponding sequence/molecule.

### Statistics

In Table 2, statistics from the analyses of the 422 datasets that belong to 1^{st} analyzed collection is described. These include starting number of sequenced reads, reads remaining after adapter removal, reads remaining after UMI removal, reads mapping to known miRNAs, reads mapping to known tRNAs, etc.

### Method for Identifying Differentially Abundant Molecules

Separately for each of the four RNA classes of interest, the present study used DESeq2 described in Love et al 2014 “Moderated estimation of fold change and dispersion for RNA-seq data with DESeq2” (https://doi.org/10.1186/s13059-014-0550-8) to identify those molecules that changed between conditions of interest.

If a patient participated in the longitudinal portion of the study, only the first sample that was collected from this patient was used to avoid counting the patient more than once. To help eliminate outlier sequencing runs, pairwise Spearman correlations using isomiR expression were calculated across the samples. The present study excluded from the DESeq2 runs those samples whose mean Spearman correlations placed them in the lowest 5 percentile range.

Differential Abundance Results—Healthy Controls vs. ALS

The present study used DESeq2 to compare the profiles of each of the four classes of interest in healthy controls (101 datasets) and ALS patients (231 datasets). The present study found a total of 1480 short RNA sequences that satisfy the above thresholds: 456 isomiRs, 40 tRFs, 143 rRFs, and 841 Not-TRF-ISO-RRE RNAs Four of the 1480 sequences appear under multiple annotations in public databases, leaving 1476 unique short RNA sequences. The four sequences are:
GCUGUGAUGGCCGAGUGG (SEQ ID NO:1222) is a tRF from tRNA^{SerCGA} and also listed as a miRNA in miRCarta Rel 1.1;
GGGGAUGUAGCUCAGUGG (SEO ID NO:1231) is a tRF from tRNA^{AtaCGC} and also listed as a miRNA in miRCarta Rel 1.1;
CGGGCCUGGUUAGUACUUGGAUGG (SEQ ID NO:32) is an rRF from 5S rRNA and also listed as a miRNA in miRCarta Rel 1.1; and,
UCGGCUGUUAACCGAAAGGUUGGUGGU (SEQ ID NO:484) is a tRF from tRNA^{AsnGTT} and also listed as a miRNA in miRCarta Rel 1.1.

Furthermore, the present study discarded molecules whose mean expression in either the Control or ALS groups was fewer than in 5 raw reads Raw reads were then normalized using DESeq2's defaults (based on median of ratios). The present study only considered differentially abundant short RNA with absolute values of log₂ fold change ≥0,4, and a corresponding False Discovery Rate (FDR) ≤5.0E-02. Additionally, the present study discarded molecules whose mean expression following DESeq2 normalization) was less than 5 in either the Control or ALS groups.

The present study used DESeq2 to compare Healthy Controls with ALS patients (independently of whether the ALS patients were treated with Riluzole or not when the sample was collected/recorded).

Of the 1480 short RNAs:
215 isomiRs increase in ALS (compared to controls)
241 isomiRs decrease in ALS (compared to controls)
13 tRFs increase in ALS (compared to controls)
27 tRFs decrease in ALS (compared to controls)
44 rRFs increase in ALS (compared to controls)
99 rRFs decrease in ALS (compared to controls)
439 “other” short RNAs increase in ALS (compared to controls)
402 “other” short RNAs decrease in ALS (compared to controls)

Table 3 shows a list of these molecules. These molecules, individually, or in combinations of two or more, can serve as biomarkers that can distinguish ALS patients from healthy individuals.

### Correlation Methods and Results

The cerebrospinal fluid neurofilament light chain (NfL) is a known quantitative biomarker of ALS aggressiveness. Similarly, the rate of disease progression rate/velocity of ALSFRS, known as “slope,” is another such quantitative measure. The present study evaluated the relevance of all four classes of molecules by computing their correlations with NfL and the slope.

For this analysis all ALS samples of the 1^{st} collection for which metadata were available were started with. Analogously to what the present study did with the differential abundance analysis, if a patient participated in the longitudinal portion of the study, only their first sample was used to avoid counting the patient more than once. A total of 248 ALS samples were used in the correlations. The present study used the same expression threshold and DESeq2 normalization thresholds as before to filter molecules.

Tables 4A and 4B summarize the results. As shown in Table 4A, 1004 comparisons (comprising 709 unique molecules) were found correlated with either the NfL or the slope measures across the 248 ALS samples. The Table shows both Spearman and Pearson correlations. The shown molecules satisfied the following criteria: Spearman correlation pVal≤0.05 and FDR≤0.05 for either Spearman of Pearson correlation. The “treatment” column indicates stratification: (a) patients are treated with Riluzole; (b) patients are not treated with Riluzole; and, (c) patients are considered regardless of treatment status. Table 4B provides additional results. In some embodiments of the methods of the present invention, the signature comprises one or more RNAs as set forth in Tables 3 or 4. In other embodiments, the signature comprises one or more RNAs as set forth in Table 3B or 4B.

### Survival Analysis Methods and Results

In this analysis the relevance of the four classes of molecules were evaluated by determining whether they can predict time-to-death. The present study sought to also perform these predictions after stratifying by treatment status (Riluzole or not) and controlling for demographic and disease characteristics.

The present study started with all diseased ALS samples for which metadata were available. Analogously to what the present study did with the differential abundance analysis, if a patient participated in the longitudinal portion of the study, only their first sample was used to avoid counting the patient more than once. A total of 248 ALS samples were used in the Cox models. The present study used the same expression threshold and DESeq2 normalization thresholds as before to filter molecules.

The present study created Cox models for each combination of:
All 4 classes of molecules;
Expression data and log-transformed expression data;
Stratification by the presence of absence of treatment with Riluzole treatment, and by ignoring the treatment status; and,
Survival from onset and survival from enrollment.

For the univariate analysis, Kaplan-Meier curves and statistics were also generated using the molecules' mean expression to form a “low expression” and a “high expression” group. Multivariate analysis was also performed to control for demographics and disease characteristics. The following separate multivariate runs were analyzed:
(a) Expression (molecule expression)+Demographics (age at onset, age at enrollment, sex); and,
(b) Expression (molecule expression)+Demographics (age at onset, age at enrollment, sex) +Disease-related information (onset type, ALSFRS, rd50).

For both the univariate and multivariate results, a molecule was deemed significant if and only if the following two criteria were met: (i) the probability of the estimated coefficient of the molecule must be ≤5.0E-02 and (ii) the pVal of one or both of the “likelihood ratio test” of “Wald test” must be ≤5.0E-02.

The short RNAs identified in the univariate analyses are set forth in SEQ ID NOs:2726-19896. The short RNAs identified in the multivariate analyses that considered expression and demographics are set froth in SEQ ID NOs:19897-40953. The short RNAs identified in the multivariate analyses that considered expression, demographics and disease-related information. are set forth in SEQ ID NOs:40954-65054. A total of 2320 short RNAs met the thresholds in the univariate analysis. For the multivariate runs, 2378 and 2509 short RNAs, respectively, satisfied the thresholds. The union of these three collections of short RNAs yielded a total of 2815 unique sequences/short RNAs.

### Notes on the Analysis of the First Collection of Datasets (Plasma Samples)

Many of the short RNAs that satisfy the various thresholds are not those encountered in public databases (e.g. the 0|0 isomiRs found in miRBase), but variants that contain e.g. post-transcriptional modifications, SNPs, or mutations.
Several of the isomiRs that emerge from the analysis are prefixed with “TJU” and refer to miRNAs that the present study identified and reported previously (in a previous patent disclosure and in Londin et al PNAS 2015). For example: a) several short RNAs from the TJD_CMC_MD2.ID03138 precursor show increased expression in ALS patients compared to controls; b) analogously, several short RNAs from the TJU_CMC_MD2.TD00898 precursor show decreased expression in ALS patients compared to controls.
Short RNAs from the two precursors mentioned in the previous bullet and from the precursors labeled TJU_CMC_MD2.ID01326 and TJU_CMC_MD2.ID02236 are statistically significant in survival analysis.
A short RNA from TJU_CMC_MD2.ID03138 is negatively correlated with NIL.
The present study found multiple examples of “groups of short RNAs” satisfying the following properties:
   They belong to the Not-TRF-ISO-RRF class;
   Each member of a given group closely resembles a known short RNA, e.g. a miRNA isoform, but differs from the known sequence at a single nucleotide;
   The location of the single nucleotide difference within the wildtype sequence (reference) is different for each member of the group at hand;
   Each modified sequence is present in multiple samples (controls and ALS patients); and
   One or more of the modified sequences belonging to a group are differentially abundant between controls and ALS patients in a statistically-significant manner.

Table 5A shows a few such groups of short RNAs. It was also noted that the present study observed molecules with these modifications in the other analyses (correlations and survival analysis).
The present study found multiple short RNAs satisfying the following properties:
   They belong to the Not-TRE-ISO-RRF class;
   They also do not map elsewhere on the human genome;
   They map exactly (zero insertions, zero deletions, zero replacements) to sequences of microbial origin.

To help identify the bacterium that could be the of these short RNAs, the present study used the public database “SILVA”—described in Glöckner et al 25 years of serving the community with ribosomal RNA gene reference databases and tools, Journal of Biotechnology. 2017. Silva contains of 16S/18S and 23S/28S rRNA sequences from bacteria, archaea and eukarya. The present study focused on reads that receive a lot of support across many of the analyzed samples and mapped them to the rRNAs contained in the silva database. The present study found that in various combinations these reads map to the rRNAs of different organisms, e.g. bacteria from the phylum Verumicrohia and bacteria from the phylum Proteobacteria. One such example is the short RNA GUAAUGGCGGGAACUCUGGACAGACUGCCU (SEQ ID NO:1194) (see Tables 5A, 5B, and 5C) that has an exact match in an rRNA sequence from Veramicrobia.

Table 5B lists 349 short RNAs that emerge as statistically significant in the analyses and have exact matches (zero insertions, zero deletions, and zero replacements) to RNA from the genus Pelomonas.

Table 5C contains 838 short RNAs that have exact matches (zero insertions, zero deletions, and zero replacements) to rRNA from the SILVA database (matching rRNA from Pelomonas and other organisms) as well as emerged from the Fisher-Exact enrichment analysis as being statistically significant differences between Controls and ALS.

Table 5D contains an additional 111 molecules from those that were analyzed using the Fisher's Exact enrichment analysis and further satisfy the following criteria: their mean unnormalized abundance in either Controls or ALS is ≥10; their FDR ≤1.0 E-09; and, they are not already included in Table 5C. Tables 5C and 5D are non-exhaustive lists.

Because of these findings, it was believed that the microbiome and its fragments may aid in the diagnosis and prognosis of ALS and could be candidates for therapeutic exploration. From a prognosis perspective, a large number of these short RNAs associate significantly with survival and correlate with NfL.

Given their properties, many of which the present study enumerated above, the short RNAs that emerge from the analyses and are listed in Tables 3, 4, 5A, 5B, 5C, 5D and 6E hold the potential to serve as diagnostic or prognostic biomarkers. For the purpose they can be used in isolation or in groups of two or more molecules. Additionally, the short RNAs hold the potential to serve as candidate therapeutic targets.

It should be noted that many of the observations that result from the analyses of the 1^{st} collection of datasets (plasma samples—Example 2) hold for the 200 collection (serum samples—Example 2) discussed below, and many short RNAs are differentially abundant between controls and ALS patients in both collections.

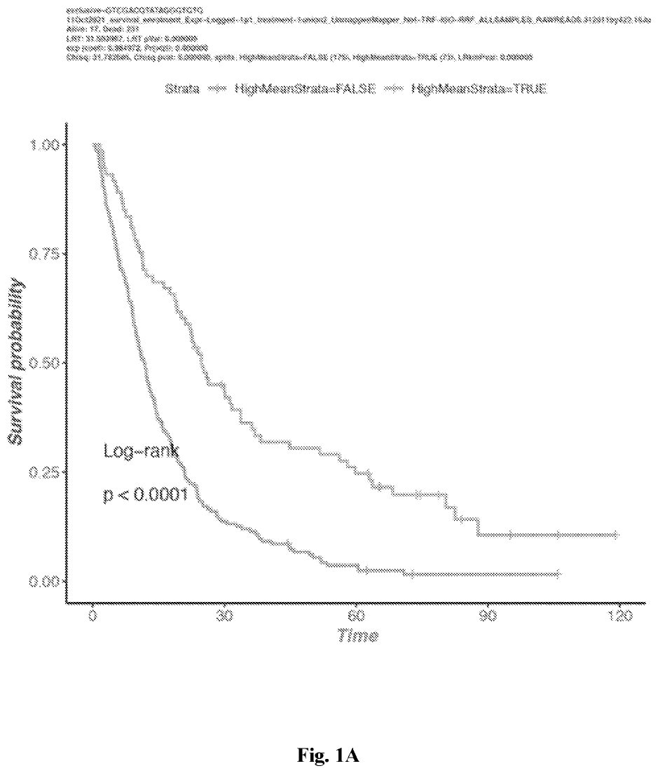

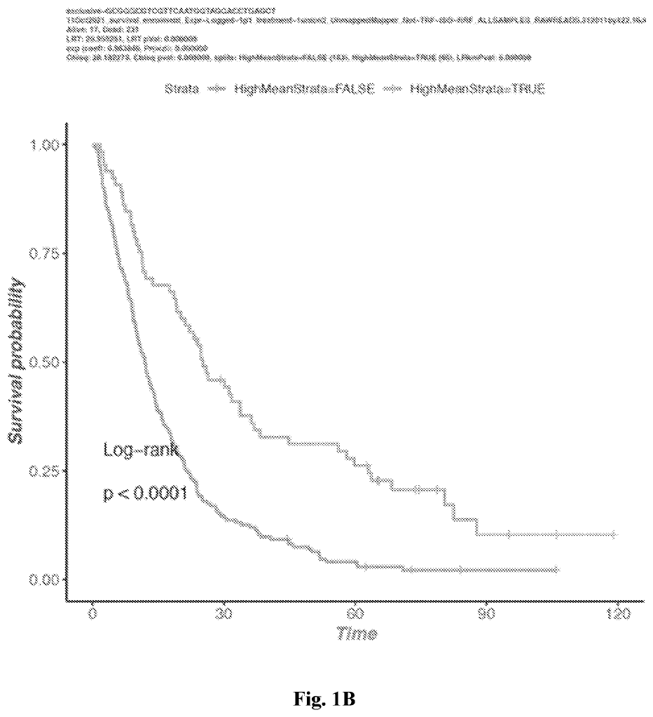

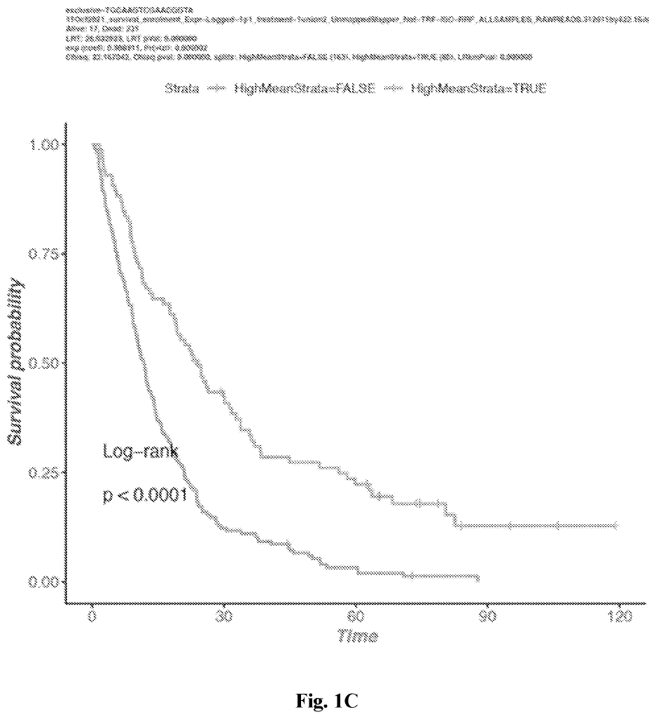

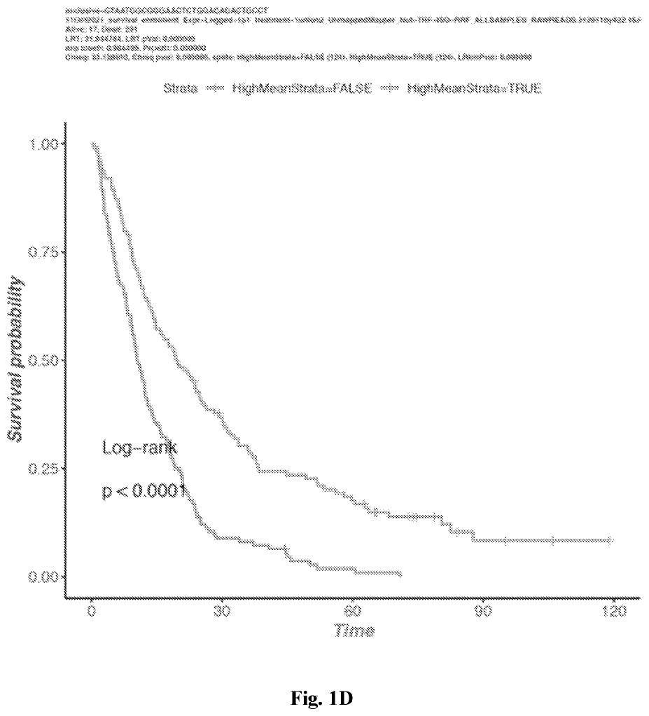

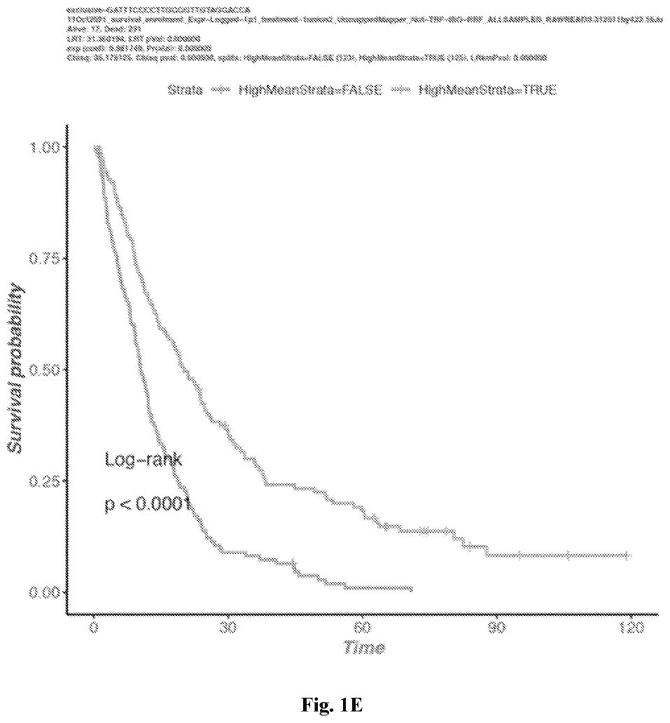

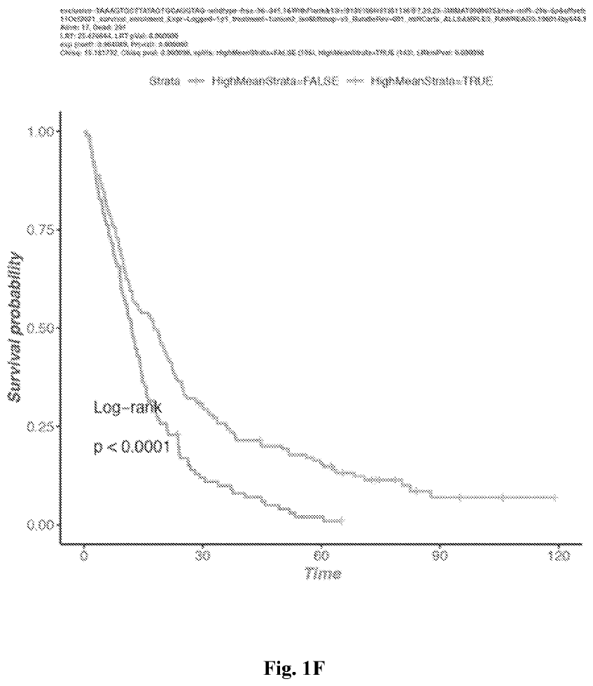

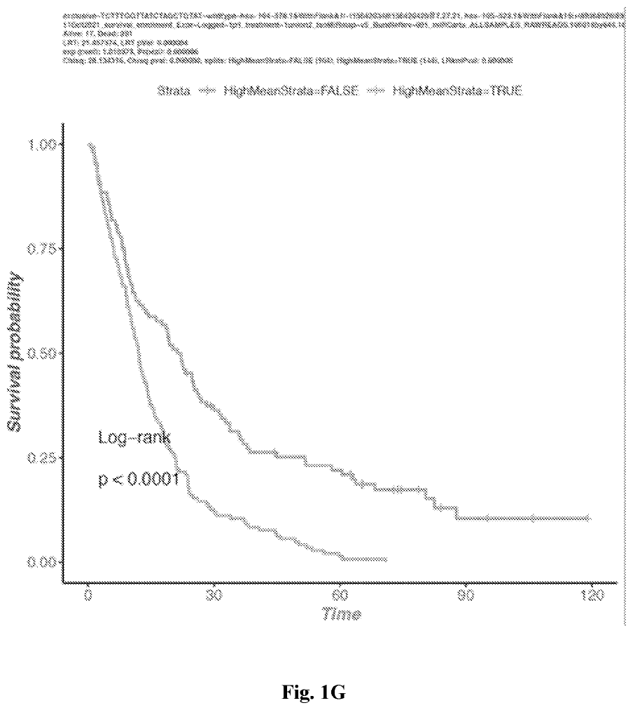

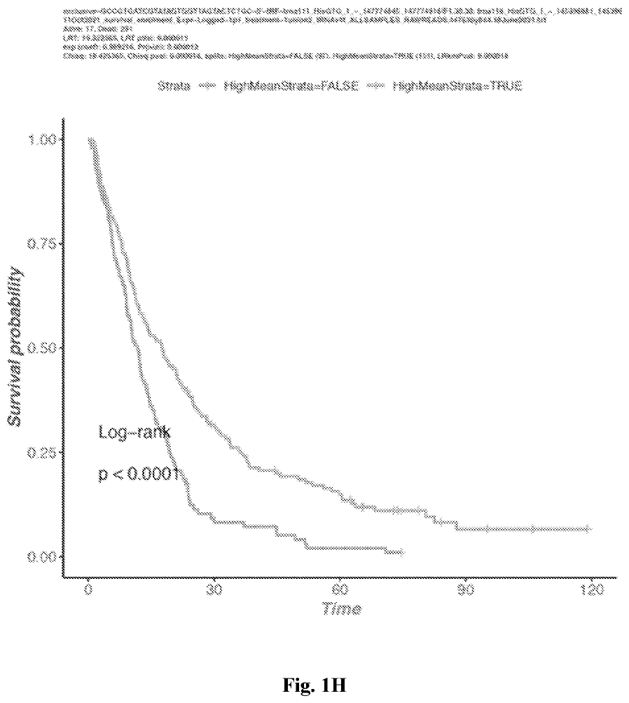

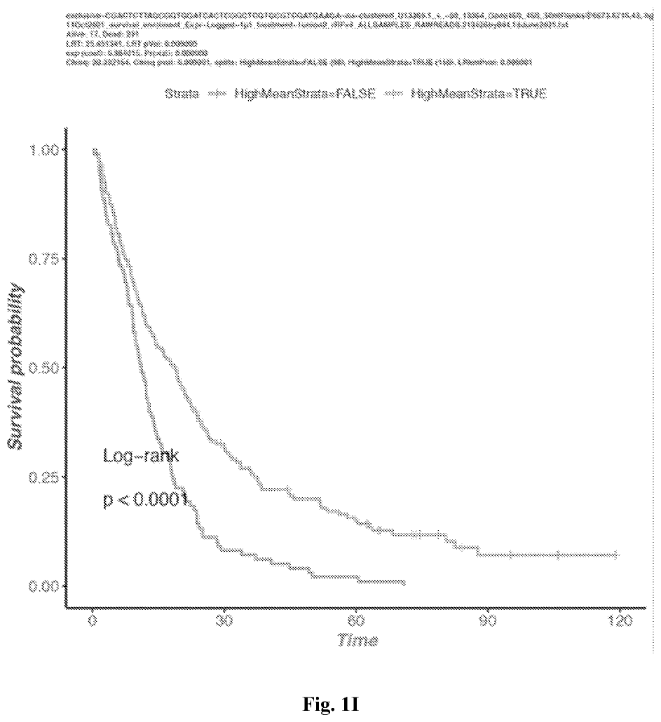

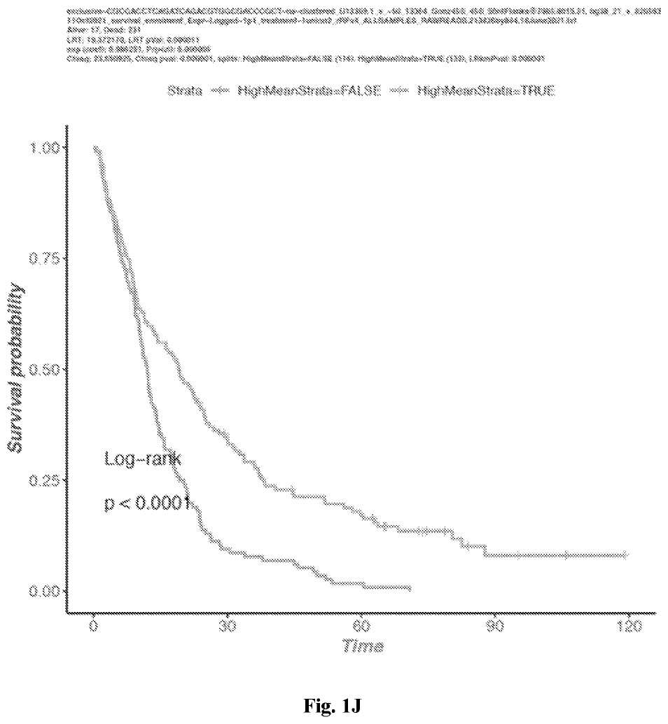

Examples of association of different short RNAs with survival: A few examples of sequences that associate with ALS patient survival in a statistically significant manner are be presented in FIGS. 1A-1J.

### Example 2: Second of the Two Collections—GSE148097

About the data

The RNA-seq data of the second collection appear on NIH's Gene Expression Omnibus (GEO) under accession number GSE148097. Table 6 lists the 19 datasets that the present study analyzed, and associated information.

### Preprocessing of The Deep Sequencing Datasets

Unlike in Example 1, in Example 2, UMI-tools were not use because UMI tags were not utilized during library construction. Instead, sequencer reads were processed using the tool cutadapt described in Martin, Marcel, Cutadapt Removes Adapter Sequences From High-Throughput Sequencing Reads, EMBNetJ, 2011 (https://doi.org/10.14806/ej.17.1.200) in order to remove the sequencing adapters. An error rate of 12% was used with a quality cutoff of 15. As before, reads were discarded for further analysis if the 3′ adapter was not identified or if they were <15 nts.

### Mining of the Deep Sequencing Datasets

The present study preprocessed each raw dataset from Table 6 as outlined above. The present study profiled the short RNA in each dataset by focusing on the same four RNA classes as with the first collection: isomiRs; tRFs; rRFs, and Not-TRF-ISO-RRF short RNAs. The present study identified and quantified the short RNAs of each class similarly to how the first collection was analyzed: isoMiRmap was used to profile isomiRs; MINTmap was used to profile tRFs; the unpublished tool MINRmap was used to profile rRFs; and those short RNAs that received above-threshold support and remained after accounting for isomiRs, tRFs, and rRFs were labeled as “Not-TRF-ISO-RRF.”

### Method for Identifying Differentially Abundant Molecules

Mirroring what was done with the first collection, for each of the four RNA classes of interest, the present study used DESeq2 to identify those molecules that changed between ALS patients and healthy Controls. Molecules were discarded if they did not have a mean expression ≥5 reads in either the healthy Control or ALS patient groups. Raw reads were then normalized using DESeq2's defaults (based on median of ratios). The present study only considered differentially abundant short RNA whose absolute value of log fold change ≥0.4, and corresponding FDR ≤5.0E-02. Molecules were again discarded that did not have at least a mean expression (DESeq2 normalization) of 5 reads in either the Control or ALS groups.

The present study used DESeq2 to compare Healthy Controls with ALS patients.

### Statistics

In Table 7, statistics from the analyses of the 19 datasets of this second collection is described. These include: starting number of sequenced reads, reads remaining after adapter removal, reads mapping to known miRNAs, reads mapping to known tRNAs, etc.

Differential Abundance Results—Healthy Controls vs. ALS

The present study used DESeq2 to compare the profiles of each of the four classes of interest in controls (6 datasets) and ALS patients (13 datasets). The present study found a total of 255 short RNA sequences that satisfy the above thresholds: 6 isomiRs, 34 tRFs, 5 rRFs, and 210 short RNAs that cannot be classified as isomiR, tRF, or rRF. One of the 253 sequences appears under multiple annotations in public databases: GGGGAUGUAGCUCAGUGGUAGA (SEQ ID NO:66693) is a tRF from tRNA^{AlaCGC} and also listed as a miRNA in miRCarta Rel 1.1. This leaves 254 unique short RNA sequences.

Of the 255 short RNAs:
3 isomiRs increase in ALS (compared to controls)
3 isomiRs decrease in ALS (compared to controls)
20 tRFs increase in ALS (compared to controls)
14 tRFs decrease in ALS (compared to controls)
2 rRFs increase in ALS (compared to controls)
3 rRFs decrease in ALS (compared to controls)
86 “other” short RNAs increase in ALS (compared to controls)
124 “other” short RNAs decrease in ALS (compared to controls)

Table 8 shows a list of these molecules. These molecules, individually, or in combinations of two or more, can serve as biomarkers that can distinguish ALS patients from healthy individuals. The molecules could also serve as candidate therapeutic targets.

### Enumerated Embodiments

In some aspects, the present invention is directed to the following non-limiting embodiments:

Embodiment 1: A method for treating or preventing amyotrophic lateral sclerosis (ALS) in a subject in need thereof, the method comprising: isolating RNAs from a sample obtained from the subject, wherein the RNAs have a length of 100 nucleotides or less; characterizing the RNAs and their relative abundance in the sample to determine a signature. wherein the signature is indicative of presence or absence of ALS; and administering to the subject a treatment for ALS if the identified signature indicates presence of ALS.

Embodiment 2: The method of Embodiment 1, wherein: (a) the signature comprises at least one RNA selected from the group consisting of RNAs set forth in SEQ ID NOs: 1-711, 1481-1545, 65683-65745, 66521-66593, 66632-66674, and 66876-66986, and an increased level of the at least one RNA in comparison to a baseline level is indicative of presence of ALS, and/or (b) the signature comprises at least one RNA selected from the group consisting of RNAs set forth in SEQ ID NOs: 712-1480, 1546-1679, 65746-66520, 66594-66631, 66675-66875, and 66987-67130 and a decreased level of the at least one RNA in comparison to a base fine is Indicative of presence of ALS.

Embodiment 3: The method of Embodiment 1 or 2, wherein the sample is isolated from a cell, tissue or body fluid of the subject.

Embodiment 4; The method of any one of Embodiments 1-3, wherein the sample is isolated from a body fluid, and the body fluid is a plasma, a serum, a cerebrospinal fluid, of combinations thereof.

Embodiment 5: The method of any one of Embodiments 1-4, wherein the treatment for ALS comprises a glutamate blocker, a muscle relaxant, a physical therapy, or combinations thereof.

Embodiment 6: The method of Embodiment 5, wherein the treatment for ALS comprises the glutamate blocker, and wherein the glutamate blocker comprises riluzole.

Embodiment 7: The method of any one of Embodiments 1-6 further comprising treating the subject for a bacterial infection by a bacterium, wherein the signature comprises at least one RNA of the bacterium.

Embodiment 8: The method of Embodiment 7, wherein the bacterium is a Verumicrobia or a Proteobacteria.

Embodiment 9: The method of any one of Embodiments 7-8, wherein the treating for the infection comprises an antibiotic.

Embodiment 10: The method of any one of Embodiments 1-9, wherein the subject is a mammal.

Embodiment 11: The method of any one of Embodiments 1-10, wherein the subject is a human.

Embodiment 12: A method for determining speed of amyotrophic lateral sclerosis (ALS) progression in a subject in need thereof, the method comprising: isolating RNAs from a sample obtained from the subject, wherein the RNAs have length of 100 nucleotides or less; characterizing the RNAs and their relative abundance in the sample to identify a signature; wherein the signature is indicative of speed of ALS progression, and determining the speed of ALS progression to the subject based on the identified signature.

Embodiment 13: The method of Embodiment 12, wherein the signature comprises at least one RNA selected from the group consisting of RNAs set forth in SEQ ID NOs: 1680-2725, and wherein an altered level of the at least one RNA in comparison to a reference level is indicative of fast ALS progression.

Embodiment 14: A method for determining a stage of amyotrophic lateral sclerosis (ALS) in a subject, the method comprising: isolating RNAs from a sample obtained from the subject, wherein the RNAs have a length of 100 nucleotides or less; and characterizing the RNAs and their relative abundance in the sample to identify a signature, wherein the signature is indicative of a stage of ALS; and determining the stage of the ALS in the subject based on the identified signature.

Embodiment 15: The method of Embodiment 14, wherein the signature comprises at least one RNA selected from the group consisting of RNAs set forth in SEQ ID NOs: 2726-65054, and wherein an altered level of the at least one RNA in comparison to a reference level is indicative of a later stage of ALS.

Embodiment 16: The method of any one of Embodiments 12-15, wherein the sample is isolated from a cell, tissue or body fluid of the subject

Embodiment 17: The method of any one of Embodiments 12-16, wherein the sample is isolated from a body fluid, and the body fluid is a plasma, a serum, a cerebrospinal fluid, or combinations thereof.

Embodiment 18: The method of any one of Embodiments 12-17, wherein the subject is a mammal.

Embodiment 19: The method of any one of Embodiments 12-18, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

Lengthy table referenced here
US20250297309A1-20250925-T00001
Please refer to the end of the specification for access instructions.

Lengthy table referenced here
US20250297309A1-20250925-T00002
Please refer to the end of the specification for access instructions.

Lengthy table referenced here
US20250297309A1-20250925-T00003
Please refer to the end of the specification for access instructions.

Lengthy table referenced here
US20250297309A1-20250925-T00004
Please refer to the end of the specification for access instructions.

Lengthy table referenced here
US20250297309A1-20250925-T00005
Please refer to the end of the specification for access instructions.

Lengthy table referenced here
US20250297309A1-20250925-T00006
Please refer to the end of the specification for access instructions.

Lengthy table referenced here
US20250297309A1-20250925-T00007
Please refer to the end of the specification for access instructions.

Lengthy table referenced here
US20250297309A1-20250925-T00008
Please refer to the end of the specification for access instructions.

Lengthy table referenced here
US20250297309A1-20250925-T00009
Please refer to the end of the specification for access instructions.

Lengthy table referenced here
US20250297309A1-20250925-T00010
Please refer to the end of the specification for access instructions.

Lengthy table referenced here
US20250297309A1-20250925-T00011
Please refer to the end of the specification for access instructions.

Lengthy table referenced here
US20250297309A1-20250925-T00012
Please refer to the end of the specification for access instructions.

Lengthy table referenced here
US20250297309A1-20250925-T00013
Please refer to the end of the specification for access instructions.

Lengthy table referenced here
US20250297309A1-20250925-T00014
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20250297309A1). An electronic copy of the table will also be available from the USPTO upon request and payment of the fee set forth in 37 CFR 1.19(b)(3).

1. A method for treating or preventing amyotrophic lateral sclerosis (ALS) in a subject in need thereof, the method comprising:
isolating RNAs from a sample obtained from the subject, wherein the RNAs have a length of 100 nucleotides or less;
characterizing the RNAs and their relative abundance in the sample to determine a signature, wherein the signature is indicative of presence or absence of ALS; and
administering to the subject a treatment for ALS if the identified signature indicates presence of ALS.

2. The method of claim 1, wherein:
(a) the signature comprises at least one RNA selected from the group consisting of RNAs set forth in SEQ ID NOs: 1-711, 1481-1545, 65683-65745, 66521-66593, 66632-66674, and 66876-66986, and an increased level of the at least one RNA in comparison to a baseline level is indicative of presence of ALS, and/or
(b) the signature comprises at least one RNA selected from the group consisting of RNAs set forth in SEQ ID NOs: 712-1480, 1546-1679, 65746-66520, 66594-66631, 66675-66875, and 66987-67130 and a decreased level of the at least one RNA in comparison to a base line is indicative of presence of ALS.

3. The method of claim 1, wherein the sample is isolated from a cell, tissue or body fluid of the subject

4. The method of claim 1, wherein the sample is isolated from a body fluid, and the body fluid is a plasma, a serum, a cerebrospinal fluid, or combinations thereof.

5. The method of claim 1, wherein the treatment for ALS comprises a glutamate blocker, a muscle relaxant, a physical therapy, or combinations thereof.

6. The method of claim 5, wherein the treatment for ALS comprises the glutamate blocker, and wherein the glutamate blocker comprises riluzole.

7. The method of claim 1, further comprising treating the subject for a bacterial infection by a bacterium, wherein the signature comprises at least one RNA of the bacterium.

8. The method of claim 7, wherein the bacterium is a Verumicrobia or a Proteobacteria.

9. The method of claim 7, wherein the treating for the infection comprises an antibiotic.

10. The method of claim 1, wherein the subject is a mammal.

11. The method of claim 1, wherein the subject is a human.

12. A method for determining speed of amyotrophic lateral sclerosis (ALS) progression in a subject in need thereof, the method comprising:
isolating RNAs from a sample obtained from the subject, wherein the RNAs have length of 100 nucleotides or less;
characterizing the RNAs and their relative abundance in the sample to identify a signature, wherein the signature is indicative of speed of ALS progression; and
determining the speed of ALS progression in the subject based on the identified signature.

13. The method of claim 12, wherein the signature comprises at least one RNA selected from the group consisting of RNAs set forth in SEQ ID NOs: 1680-2725, and wherein an altered level of the at least one RNA in comparison to a reference level is indicative of fast ALS progression.

14. A method for determining a stage of amyotrophic lateral sclerosis (ALS) in a subject, the method comprising:
isolating RNAs from a sample obtained from the subject, wherein the RNAs have a length of 100 nucleotides or less; and
characterizing the RNAs and their relative abundance in the sample to identify a signature, wherein the signature is indicative of a stage of ALS; and
determining the stage of the ALS in the subject based on the identified signature.

15. The method of claim 14, wherein the signature comprises at least one RNA selected from the group consisting of RNAs set forth in SEQ ID NOs: 2726-65054, and wherein an altered level of the at least one RNA in comparison to a reference level is indicative of a later stage of ALS.

16. The method of claim 12 wherein the sample is isolated from a cell, tissue or body fluid of the subject

17. The method of claim 12, wherein the sample is isolated from a body fluid, and the body fluid is a plasma, a serum, a cerebrospinal fluid, or combinations thereof.

18. The method of claim 12, wherein the subject is a mammal.

19. The method of claim 12, wherein the subject is a human. 52380128.1
